(19) United States
(12) Patent Application Publication
(10) Pub. No.: US 2018/0353613 A1
(43) Pub. Date: Dec. 13, 2018

(54) ANIONIC NANOPARTICLES FOR USE IN THE DELIVERY OF ANIONIC SMALL MOLECULE DRUGS

(71) Applicant: B.G. NEGEV TECHNOLOGIES AND APPLICATIONS LTD, Beer Sheva (IL)

(72) Inventors: Smadar COHEN, Beer Sheva (IL); Olga KRYUKOV, Beer Sheva (IL); Efrat FORTI, Beit-Kama (IL); Emil RUVINOV, Arad (IL)

(21) Appl. No.: 15/571,330

(22) PCT Filed: May 4, 2016
(86) PCT No.: PCT/IL2016/050467

§ 371 Date: Nov. 2, 2017

Related U.S. Application Data
(60) Provisional application No. 62/157,007, filed on May 5, 2015.

(51) Int. Cl. A61K 47/69; A61P 35/00; A61K 31/519; A61K 31/704; A61K 47/52; A61K 47/61

(52) U.S. Cl. CPC A61K 47/6939; A61P 35/00; A61K 47/61; A61K 31/704; A61K 47/52; A61K 31/519

(57) ABSTRACT

The present invention provides an anionic nanoparticle formed from an anionic polymer and an anionic small molecule drug and further comprising a cation, wherein said anionic polymer is selected from an anionic natural polysaccharide or a derivative thereof, and an anionic synthetic polymer. The present invention further provides uses of the anionic nanoparticle for the deliver} 7 of the anionic small molecule drugs into cells and in methods for treating a disease, disorder or condition selected from cancer, metabolic, neurodegenerative, cardiovascular, infectious and inflammatory diseases or disorders, and methods for preparation of the nanoparticle. The present invention also provides a nanoparticle comprising a divalent cation and an anionic small molecule drug and lacking an anionic polymer, and methods for its production.

### FIELD OF THE INVENTION

The present invention relates to nanoparticles for delivery of anionic small molecule drugs.

### BACKGROUND OF THE INVENTION

Small molecule drugs are rapidly cleared from circulation and only low doses of the administered drug reach and accumulate in target sites, such as tumor vicinity, whereas the localization in healthy organs (liver, spleen, kidneys, lungs and bone marrow) is relatively high, leading to adverse side effects of these drugs. The entrapment of small drugs in nanoparticles (NPs) essentially aims to increase the circulation time of the drug and enable its accumulation at target sites (Blanco et al., 2015).

In case of solid tumors, passive targeting of NPs at tumor vicinity occurs via the Enhanced Permeability and Retention (EPR) mechanism effect. The EPR is a pathophysiological property of solid tumor, manifested by a significant increase in vascular permeability together with the ineffective lymphatic drainage of solid tumors. EPR allows nanoparticles (NPs) with sizes up to several hundreds of nanometers to accumulate in tumors over time (Maeda et al., 2013).

For other target sites such as in different diseases or in tumors, where EPR effect is not a factor, strategies for active drug targeting were developed. These strategies rely on the use of targeting agents on the NPs, such as proteins (mainly antibodies and their fragments), nucleic acid (aptamers) or other receptor ligands (peptides, carbohydrates), which specifically bind to receptors or antigens uniquely expressed (or overexpressed) at the target site. The targeted NPs reach and accumulate at the target site and there, several mechanisms can occur by which the drug reaches the cells (Bertrand et al., 2014). In one option, the drug is released from NPs outside the cells and then it diffuses into the cells. In a second option, the NPs are taken up by cells and release the drug intracellularly.

The NPs developed so far for both passive and active drug delivery are in the form of long-circulating liposomes (Barenholz, 2012), micelles, and as polymeric particles (Blanco et al., 2015). Each of these carriers has advantages and drawbacks. The main challenge in all of these carriers is their fabrication in the format appropriate for a systemic and intracellular delivery.

For systemic and intracellular delivery, the particle size should be 100 nm (corresponding to size of NPs) and the collection of particles should be mono-disperse, thus presenting a great challenge in most of the technologies available for NP fabrication (Blanco et al., 2015).

### SUMMARY OF THE INVENTION

It has been found in accordance with the present invention, that anionic polymers are capable of functioning as carriers for the delivery of anionic small molecule drugs into cells by forming nanoparticle complexes with the anionic small molecule drugs via electrostatic interactions with calcium ions.

Accordingly, in one aspect the present invention provides an anionic nanoparticle formed from an anionic polymer and an anionic small molecule drug and further comprising a cation, wherein said anionic polymer is selected from an anionic natural polysaccharide or a derivative thereof, and an anionic synthetic polymer.

In another aspect, the present invention provides the use of the anionic nanoparticle of the invention as defined above, for the delivery of the anionic small molecule drug into cells.

In a further aspect, the present invention provides a pharmaceutical composition comprising the anionic nanoparticle of the invention as defined above and a pharmaceutically acceptable carrier.

In an additional aspect, the present invention provides an anionic nanoparticle of the invention as defined above or a pharmaceutical composition as defined above for use in a method of treating a disease, disorder or condition selected from cancer such as colon cancer, ovarian carcinoma, and breast cancer, or metabolic, neurodegenerative, cardiovascular, infectious or inflammatory diseases or disorders.

In another aspect, the present invention provides a method for the preparation of the anionic nanoparticle of the invention as defined above, comprising mixing said anionic small molecule drug with a salt of a divalent cation that is a strong electrolyte in zwitterionic buffer at physiological pH, and adding said anionic polymer.

In yet another aspect, the present invention provides an anionic nanoparticle comprising a divalent cation and an anionic small molecule drug and lacking an anionic polymer, wherein said nanoparticle is in the form of nanoparticles capable of forming a colloidal suspension.

In a further aspect, the present invention provides a method for producing the nanoparticle lacking an anionic polymer as defined above, comprising mixing said anionic small molecule drug with a salt of a divalent cation that is a strong electrolyte in zwitterionic buffer at physiological pH.

In another aspect, the present invention provides a kit for use in delivery of anionic small molecule drugs to cells, said kit including a first container comprising an anionic polymer, a second container comprising a strong electrolyte, a third container comprising an anionic small molecule drug for delivery into cells, and a leaflet with instructions for mixing said ingredients.

### BRIEF DESCRIPTION OF DRAWINGS

FIGS. 1A-1B show complex size (evaluated by DLS) as a function of calcium ion and MTX concentration, and time (left to right—0, 24 or 48 hours), without (A) or with HAS (B). All differences were not significant. Concentrations of MTX and Ca²⁺ in A and B (left to right each time point): 10 pg/ml MTX/1 mM Ca²⁺, 10 pg/ml MTX/5 mM Ca²⁺, 100 pg/ml MTX/1 mM Ca²⁺, 100 pg/ml MTX/5 mM Ca²⁺, 1 ng/ml MTX/1 mM Ca²⁺, 1 ng/ml MTX/5 mM Ca²⁺. In B—concentration of HAS is 0.5 μg/ml.

FIGS. 2A-2B show surface charge (potential) of MTX complexes as a function of calcium ion and MTX concentrations, and time (left to right—0, 24 or 48 hours), without (A) or with HAS (B). *—p<0.05 (Sidak's multiple comparisons test). Concentrations of MTX and Ca²⁺ in A and B (left to right each time point): 10 μg/ml MTX/1 mM Ca²⁺, 10 μg/ml MTX/5 mM Ca^{2+,} 100 μg/ml MTX/1 mM Ca²⁺, 100 μg/ml MTX/5 mM Ca²⁺, 1 ng/ml MTX/1 mM Ca², 1 ng/ml MTX/5 mM Ca²⁺. In B—concentration of HAS is 0.5 μg/ml.

FIGS. 3A-3D show dry transmission electron microscopy of MTX complexes. A. Ca²⁺-MTX complex; B. HAS-Ca²⁺-MTX complex. C and D correspond to A and B, respectively, at a higher magnification.

FIGS. 4A-4B show the viability profile of CT26 mouse colon carcinoma treated with free MTX (circles), Ca²⁺-MTX complex (squares), or HAS-Ca²⁺-MTX complex (triangles). B is a blow-up of the region in A corresponding to MTX concentrations of 10⁻⁵-10⁻³ μg/ml.

FIG. 5 shows the viability profile of MDA-MB-231 cells after treatment with free MTX (filled circles) or AlgS-Ca²⁺-MTX (empty squares) complexes. P (interaction, 2-way ANOVA<0.0001, *p<0.05).

FIGS. 6A-6B show physicochemical characterization of various formulations of AlgS-Ca²⁺-DOX complexes (according to Table 3). A. Complex diameter (in nm) of various formulations as measured by DLS. B. Surface charge (ζ potential in mV) of various formulations. DOX concentrations in A and B (μg/ml, left to right): 5.8e⁻⁰⁰⁷, 5.8e⁻⁰⁰⁶, 5.8e⁻⁰⁰⁵, 5.8e⁻⁰⁰⁴, 5.8e⁻⁰⁰³, 0.058, 0.58.

FIGS. 7A-7B show dry transmission electron microscopy of AlgS-Ca²⁺-DOX complexes. A and B are two representative images.

FIG. 8 shows viability profile of MDA-MB-231 cells after treatment with free DOX (filled circles) or AlgS-Ca²⁺-DOX (empty squares) complexes. P (interaction, 2-way ANOVA<0.0001, *p<0.05).

FIG. 9 shows viability profile of NAR cells after treatment with free DOX (filled circles) or AlgS-Ca²⁺-DOX (empty squares) NP formulations. P (interaction, 2-way ANOVA=0.0013, *p<0.05).

FIGS. 10A-10D show an analysis of DOX cellular uptake in MDA-MB-231 at 4 h (A, C) and 24 h (B, D) post transfection. Imaging flow cytometry analysis of Free DOX (A, B) or AlgS-Ca²⁺-DOX NPs uptake (C, D), percentage indicates DOX-positive cells from total cell population as obtained from the histograms of DOX intensity.

FIGS. 11A-11D show an analysis of DOX cellular uptake in NAR cells 4 h (A, C) and 24 h (B, D) post transfection. Imaging flow cytometry analysis of Free DOX (A, B) or AlgS-Ca²⁺-DOX NPs uptake (C, D), percentage indicates DOX-positive cells from total cell population as obtained from the histograms of DOX intensity.

### DETAILED DESCRIPTION OF THE INVENTION

The present invention overcomes the size and mono-dispersity challenges of fabrication for systemic and intracellular delivery by developing spontaneously assembled nanoparticles (NPs), formed due to reversible association between anionic polysaccharide and anionic drug molecules, mediated by cation bridges. These NPs have additional advantages: 1) a simple preparation method at aqueous conditions (“green technology”) is important for NP scalable production; 2) having functional carboxylates, so that targeting moieties (peptides, antibodies, receptors) can be attached onto their surface for the purpose of their targeting to cells/organs and enhancing NP penetration into cells; and 3) the relative negative surface charge makes these NPs bio-compatible, nontoxic and less amenable to opsonization and removal from circulation.

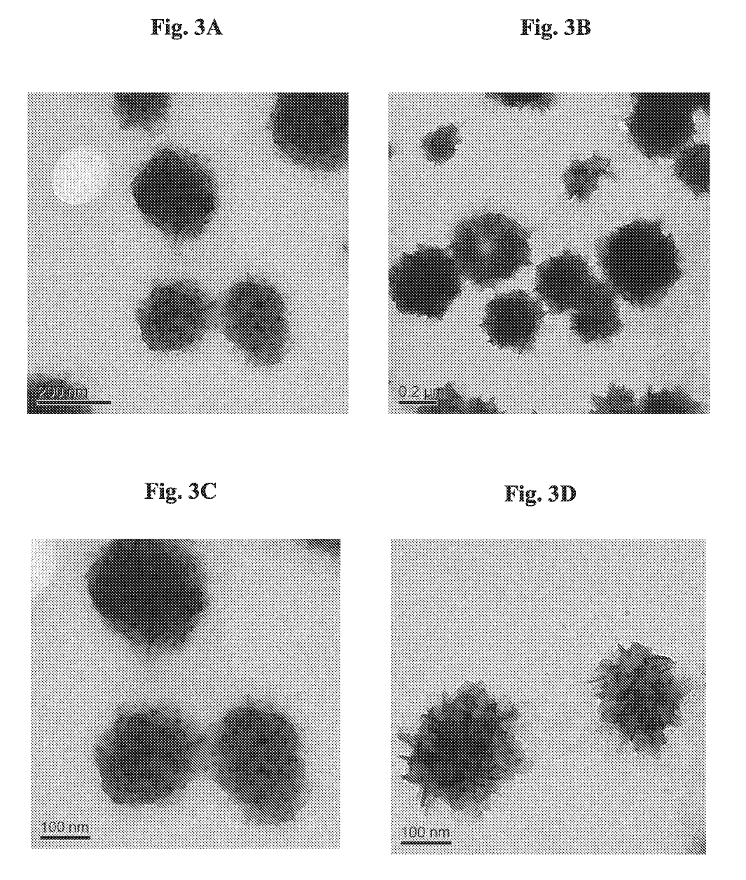

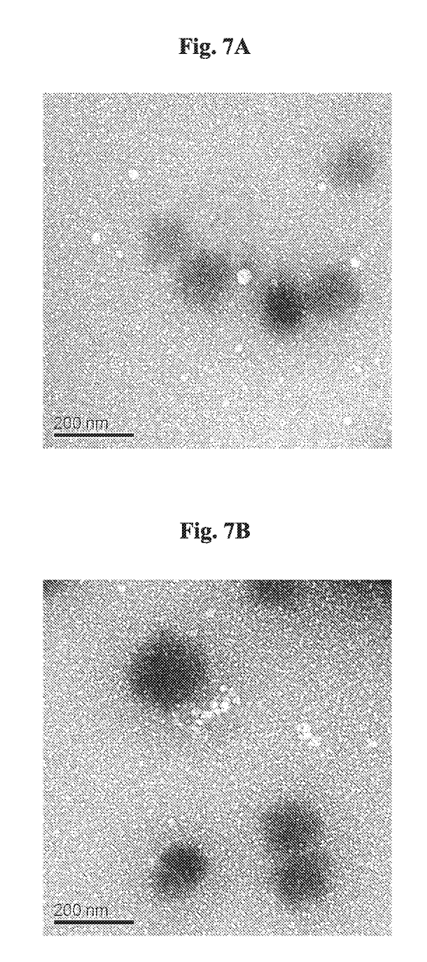

In the experiments demonstrated in the present application (see Examples 1 and 4, FIGS. 3 and 7), almost spherical complexes in the size of nanoparticles are formed from the anionic polymeric carriers and the anionic small molecule drugs therapeutics, and the interaction between the anionic small molecule drug therapeutics and the anionic polymer carriers is mediated by electrostatic interactions with cations such as calcium ions.

Since the complexes of the invention are in the size of nanoparticles, the terms “nanoparticle” and “complex” are used in the present invention interchangeably and mean particles of a size up to 300 nm.

In view of the above, in one aspect, the present invention provides an anionic nanoparticle formed from an anionic polymer and an anionic small molecule drug and further comprising a cation, wherein said anionic polymer is selected from an anionic natural polysaccharide or a derivative thereof, and an anionic synthetic polymer. It follows that the anionic complex of the invention comprises an anionic polymer, an anionic small molecule drug and a cation.

The term “anionic nanoparticle” or “anionic complex” as used herein means a nanoparticle or a complex having a negative surface charge at physiological pH.

Anionic polymers according to the invention are natural or synthetic polymers which have a net negative charge at physiological pH, i.e. between pH of 7.2 and 7.5, more specifically between pH of 7.3 and 7.4.

Anionic natural polysaccharides according to the invention include (but are not limited to) hyaluronan (HA), alginate (Alg) and their derivatives such as HA-sulfate (HAS) and Alg-sulfate (AlgS), exemplified in the present application. Synthetic anionic polymers include polyesters such as poly(lactic-co-glycolic acid), poly(lactic acid) or polycaprolactone; poly(amino acids) such as poly(glutamic acid); poly(anhydride)s; poly(sodium styrene sulfonates); poly(acrylate)s; and poly(phosphazene)s. Additional anionic polymers that can be used with the invention include anionic proteins.

In some embodiments, the anionic polymer is selected from hyaluronan (HA), alginate (Alg), HA-sulfate (HAS) and Alg-sulfate (AlgS).

In some embodiments, the molecular weight of the HA, HAS, Alg or AlgS is between 10 and 200 kDa.

Some anionic polymers have inherent biological activity or binding specificity in the human body and this can enhance their targeting and uptake by certain cells, for example, hyaluronic acid. Hyaluronic acid receptors play important biological roles in endocytosis and signal transduction. Cluster determinant 44 (CD44), receptor for hyaluronic acid-mediated motility (RHAMM), and lymphatic vessel endothelial hyaluronan receptor-1 (LYVE-1) have been identified as hyaluronic acid receptors for various biological functions.

By contrast, alginate and AlgS are plant-derived anionic polymers which do not have biological specificity in the human body and thus can be used as blank canvas on which specific groups, which are recognized in the human body, can be conjugated, or modified.

Additionally, the anionic polymers of the invention carry inherent functional carboxylates, so that various targeting moieties (peptides, antibodies, receptors) can be attached to their surface, for the purpose of targeting the complexes to their target cells (cancer cells, metastases, cells of the immune systems, etc).

Therefore, the anionic nanoparticle complex of the present invention may comprise a targeting moiety, such as a ligand to a receptor expressed on target sites. Examples for ligands that can be used for targeting of the complex of the present invention to cells or tissues of interest include, for example peptides containing RGD (Arginine-Glycine-Aspartic acid) sequence for binding to specific integrin receptors, growth factor receptors ligands such as EGF and TGFα or functional fragments thereof, antibodies or antigen-binding fragments thereof, e.g. to tumor-associated antigens, carbohydrates, such as acetylgalactosamine, a highly efficient ligand for the asialoglycoreceptors on hepatocytes, and nucleic acid aptamers.

According to the present invention, the term “a targeting moiety” does not encompass the inherent binding specificity in the human body of unmodified polymers.

In addition to their proven biocompatibility, the anionic polymeric carriers—small molecule drug complexes of the invention have additional advantages; for example, the simple preparation method at aqueous conditions (“green technology”) is important for mass production of these carriers.

The cation forming part of the complex may be a divalent cation or a multivalent cation. For example, the cation may be a divalent cation, such as Ca²⁺, Ba²⁺, Mg²⁺, Mn²⁺, Cu²⁺, Ni²⁺, Zn²⁺ or Fe²⁺; or the cation may be a multivalent cation, such as Fe³⁺, Mn⁺ or Mn⁴⁺. The cation functions as an ion bridge between the negatively charged anionic small molecule and the negatively charged anionic polymer to form the complex, i.e. the complexing between the anionic small molecule drug and the anionic polymer is mediated by electrostatic interactions with the cations. The interaction with the cation may be in the form of a cation bridge. In particular, the divalent cation is Ca²⁺, and the complexing between the anionic small molecule drug and the anionic polymer is mediated by electrostatic interactions with calcium ions. In certain complexes of the present invention, the cation forming part of the complex is not multivalent. In certain embodiments, the calcium cation is not in the form of calcium phosphate. In certain embodiments, the cation is provided as a salt that is a strong electrolyte, i.e. it is substantially dissociated in aqueous solution. For example, the electrolyte may have a degree of dissociation that is close to 1. In certain embodiments the cation is Ca²⁺. In certain embodiments the salt is CaCl₂.

In some embodiments, the nanoparticle of the invention does not comprise a positively charged polymer at physiological pH.

The anionic small molecule drug being part of the complex described above is a low molecular weight (<900 daltons) organic compound that may help regulate a biological process. The anionic small molecule drug may be selected from methotrexate (MTX), doxorubicin (DOX), carboxylate derivatives of taxol and camptothecin, flavopiridol, imatinib, phenobarbital and barbituric acid, valproate, furosemide, salicylate, acetylsalicylate, probenecid, bumetanide, piroxicam, azidodeoxythymidine, benzylpenicillin, AMD3100 (plerixafor) and an alkyl sulfonate, such as busulfan.

The anionic small molecule drug may be further selected from 1-dopa, angiotensin-converting enzyme inhibitors such as: benazeprilat, captoprilat, enalaprilat, fosinoprilat, lisinoprilat, perindoprilat, ouinaprilat, ramiprilat, spiraprilat, trandolaprilat and moexTprilat; cephalosporin; antibiotics such as: cefaclor, cefadroxil, cefamandole, cefatrizine, cefazedone, cefazuflur, cefazolin, cefbuperazone, cefclidine, cefepime, cefetecol, cefixime, cefluprenam, cefmenoxime, cefmetazole, cefodizime, cefonicid, cefoperazone, ceforanide, cefotaxime, cefotefan, cefotiam, cefoxitin, cefpimizole, cefpirome, cefoselis, cefozopran, cefpirome, cefquinome, cefpodoximc, cefroxadine, cefsulodin, cefbiramide, ceftazidime, ceftezole, ceftizoxime, cefiriaxone, cefuroxime, cephacetrile, cephalexin, cephaloglycin, cephaloridtne, cephalosporin, cephanone, cephradine, and latamoxef; penicillins such as amoxycillin, ampicillin, apalcillin, azidocillin, azlocillin, benzylpencillin, carbenicillin, carfecillin, carindacillin, cloxacillin, cyclacillin, dicloxacillin, epicillin, flucloxacillin, hetacillin, methicillin, mezlocillin, nafcillin, oxacillin, phenethicillin, piperacillin, sulbenicllin, temocillin, and ticarcillin; carbapenems; a class of beta-lactam antibiotics such as: imipenem, meropenem, ertapenem, faropenem, doripenem, danipenem/betamipron; tazobactam which inhibits the action of bacterial beta-lactamases extending the spectrum of beta-lactam antibiotics; thrombin inhibitors such as argatroban, melagatran, and napsagatran; influenza neuraminidase inhibitors such as zanamivir, peramivir and oseltamivir, non-steroidal antiinflammatory agents such as acametacin, alclofenac, alminoprofen, aspirin acetylsalicylic acid), 4-biphenylacetic acid, bucloxic acid, carprófen, cinchofen, cinmetacin, clometacin, clonixin, diclenofac, diflunisal, etodolac, fenbufen, fenclofenac, fenclosic acid, fenoprofen, ferobufen, flufenamic acid, flufenisal, flurbiprofm, fluprofen, flutiazin, ibufenac, ibuprofen, indomethacin, indoprofen, ketoprofen, ketorolac, lonazolac, loxoprofen, meclofenamic acid, mefenamic acid, 2-(8-methyl-10,11-dihydro-11-oxodibenz[b,f]oxepin-2-yl)propionic acid, naproxen, nifluminic acid, O-(carbamoylphenoxy)acetic acid, oxoprozin, piqprofen, prodolic acid, salicylic acid, salicylsalicylic acid, sulindac, suprofen, tiaprofenic acid, tolfenamic acid, tolmetin and zopemirac; prostaglandins such as ciprostene, 16-deoxy-16-hydroxy-16-vinyl prostaglandin E₂, 6,16-dimethylprostaglandin E₂, epoprostostenol, meteneprost, nileprost, prostacyclin, prostaglandins Ei, E₂, or F_{2α}, and thromboxane A₂; quinolone and fluoroquinolone antibiotics such as: acrosoxacin, cinoxacin, ciprofloxacin, enoxacin, fleroxacin, flumequine, gatifloxacin, gemifloxacin, grepafloxacin, levofloxacin, lomefloxacin, moxifloxacin, naladixic acid, norfloxacin, ofloxacin, oxolinic acid, pefloxacin, pipemidic acid, piromidic acid, prulifloxacin, rufloxacin, rosoxacin, sitafloxacin, sparfloxacin, temafloxacin, and trovafloxacin; other antibiotics such as aztreonam, imipenem, meropenem, and related carbopenem antibiotics; anticonvulsants such as clorazepate, gabapentin and valproic acid; meglitinides such as: nateglinide, repaglinide, and mitiglinide; diuretics; statins such as: atorvastatin, cerivastatin, fluvastatin, in acid, mevastatin acid, pitavastatin, pravastatin acid, rosuvastatin and simvastatin acid; antihypertensive such as: hydralazine; antimetabolites such as: pemetrexed, calcium channel blockers such as nicardipine; bisphosphonates such as: pamidronic acid, alendronic acid, ibandronic acid, risedronic acid, zoledronic acid etidronic acid, clodronic acid and tiludronic acid; immunosuppressive agents such as: mycophenolic acid; anticancer agents such as: etoposide phosphate, melphalan, and pemetrexed; angiotensin II receptor antagonists such as: candesartan, telmisartan and valsartan; antifibrinolytic agents like aminocaproic acid; acetohydroxamic acid, which is prescribed to decrease urinary ammonia, and may help antibiotics to work or help with other kidney stone treatments; verteporfin, which is a medication used as photosensitizer for photodynamic treatment to eliminate the abnormal blood vessels in the eye; Liothyronine which is a thyroid hormone drug used to treat hypothyroidism; cromolyn used in an oral form to treat mastocytosis, dermatographic urticaria and ulcerative colitis; penicillamine which is used as a form of immunosuppression to treat rheumatoid arthritis and as a chelating agent in the treatment of Wilson's disease; dimercaptosuccinic acid used as a heavy metal chelating agent; ethacrynic acid which is used as a loop diuretic medication; montelukast which is an oral leukotriene receptor antagonist (LTRA) for the maintenance treatment of asthma and to relieve symptoms of seasonal allergies; misoprostol acid which is used for the treatment and prevention of stomach ulcers, to induce labor and as an abortifacient; phosphate and phosphonate containing existing drugs illustratively include: antiviral compounds including adefovir, cidofovir, cyclic cidofovir, foscarnet, and tenofovir.

In certain embodiments, the anionic small molecule drug of the invention is methotrexate (MTX) or doxorubicin (DOX).

According to some embodiments, the anionic nanoparticle of the invention is selected from an Alg-Ca²⁺-MTX nanoparticle, an AlgS-Ca²⁺-MTX nanoparticle, a HA-Ca²⁺-MTX nanoparticle, a HAS-Ca²⁺-MTX nanoparticle, an Alg-Ca²⁺-DOX nanoparticle, an AlgS-Ca²⁺-DOX nanoparticle, a HA-Ca²⁺-DOX nanoparticle, and a HAS-Ca²⁺-DOX nanoparticle

In some embodiments, the anionic nanoparticle of the invention is a HAS-Ca²⁺-MTX nanoparticle, an AlgS-Ca²⁺-MTX, or an AlgS-Ca²⁺-DOX nanoparticle.

The molar ratio of anionic polymer to anionic small molecule drug may vary depending on the molecular weight of the anionic polymer, and may be between 100:1 and 0.01:1, between 50:1 and 0.01:1, between 20:1 and 0.01:1, between 18:1 and 0.01:1, between 16:1 and 0.01:1, between 14:1 and 0.01:1, between 12:1 and 0.01:1, between 10:1 and 0.01:1, between 8:1 and 0.01:1, between 6:1 and 0.01:1, between 4:1 and 0.01:1, or between 2:1 and 0.01:1, between 10:1 and 0.05:1, between 5:1 and 0.05:1, between 3:1 and 0.05:1, between 1:1 and 0.05:1, between 10:1 and 1:1, between 5:1 and 1:1, or between 3:1 and 1:1, or said ratio of anionic polymer to RNA or of RNA to anionic polymer is 100:1, 50:1, 20:1, 18:1, 16:1, 14:1, 12:1, 10:1, 8:1, 6:1, 4:1, 2.5:1, 2:1, 1:1, 0.8:1, 0.4:1, 0.25:1, 0.1:1 or 0.08:1.

The total concentration of bound and free Ca²⁺ may vary between 0.5-10 mM, in particular above 3 mM, depending, inter alia, on the cell type targeted for introduction of the complexes. In some embodiments, the final concentration of Ca²⁺ is about 5 mM.

The term “about” as used herein, means that values that are 10% or less above or below the indicated values are also included.

In certain embodiments, the diameter of the complex is in the range of between 50-250 nm or between 70-150 nm. In some embodiments the diameter of the complex is about 100 nm.

In some embodiments, the surface charge (or zeta potential) of the complex is negative at physiological pH.

All of the features and embodiments described above in the context of the first aspect also apply to each of the aspects described below.

In another aspect, the present invention provides a pharmaceutical composition comprising the nanoparticle of the present invention as defined hereinabove and a pharmaceutically acceptable carrier.

Pharmaceutical compositions for use in accordance with the present invention may be formulated in a conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. In certain embodiments, the pharmaceutical composition is adapted for oral administration.

The term “carrier” in the context of a pharmaceutical composition refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

In a further aspect, the present invention is directed to the use of an anionic nanoparticle as defined above for the delivery of anionic small molecule drugs to cells. For this purpose, the anionic polymer may be in a complex with a cation and the anionic small molecule drug to be delivered to the cells. The nature of the anionic polymer and the cation of this aspect are as defined above in the context of the complex.

In still another aspect, the present invention provides a kit for use in delivery of anionic small molecule drugs to cells, said kit including a first container comprising an anionic polymer as defined above, a second container comprising a strong electrolyte as defined below (such as, e.g. CaCl₂), a third container comprising a desired anionic small molecule drug for delivery into cells, and a leaflet with instructions for mixing said ingredients.

The cells, to which the anionic small molecule drug is delivered according to any one of the different aspects of the present invention, may be selected from cells in culture, either adherent to a substrate or in suspension, or cells in a living tissue such as solid tissue and blood, i.e., cells that are part of a living organism. These cells may be diseased cells, such as cancer cells, and therefore, the anionic complex of the present invention may be useful in gene therapy, for example, wherein the gene therapy comprises controlling the expression level of a gene. The cells can further be selected from various types of cells, such as immune cells, skin cells, stem cells, nerve cells, muscle cells or endothelial cells.

Thus, the anionic nanoparticle of the present invention may be for use in the treatment of any disease, disorder or condition that can be treated by administering an anionic small molecule drug. Small molecule drugs are abundant and used for treating a variety of diseases, disorders or conditions. For example, cytotoxic small molecule drugs such as MTX, DOX, taxol, and busulfan are used to treat cancer; phenobarbital and valproate are used for treating seizures such as in epilepsy; furosemide and bumetanide are used in treating heart failure; salicylate reduces aches and pains and fever, probenecid is used for treating gout and hyperuricemia; piroxicam is used as an anti-inflammatory drug; azidodeoxythymidine used as an anti-retroviral drug for preventing and treating HIV; and benzylpenicillin used as an antibiotic. Examples of additional drugs that may be used in accordance with the present invention are listed above in the context of the nanoparticles.

Such diseases disorders or conditions are therefore selected from cancer such as colon cancer, ovarian carcinoma, and breast cancer, or metabolic, neurodegenerative, cardiovascular, infectious or inflammatory diseases or disorders.

The present invention further contemplates the use of each one of its different aspects for controlling cell behavior and fate, pluripotency, differentiation, morphology, etc.

In yet another aspect, the present invention is directed to the use of an anionic nanoparticle as defined above, for sustained release of the anionic small molecule.

In an additional aspect, the present invention provides a method for treatment of a disease, disorder or condition in a subject in need thereof, comprising administering to said subject an anionic nanoparticle or the pharmaceutical composition as defined herein above.

The term “treating” or “treatment” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

In another aspect, the present invention provides an anionic nanoparticle or a pharmaceutical composition as defined herein above for treating a disease, disorder or condition in a subject in need.

In yet another aspect, the present invention provides the use of the anionic nanoparticle or the pharmaceutical composition as defined herein above for the preparation of a medicament for treating a disease, disorder or condition in a subject in need.

In some embodiments, the disease or disorder or condition is selected from colon cancer, ovarian carcinoma, and breast cancer, or metabolic, neurodegenerative, cardiovascular, infectious or inflammatory diseases or disorders. In some embodiments the disease disorder or condition is cancer.

According to a further aspect, the present application provides a method for the preparation of the anionic nanoparticle of the present invention, comprising mixing said anionic small molecule drug with a salt of a divalent cation that is a strong electrolyte in zwitterionic buffer at physiological pH, and adding said anionic polymer.

In certain embodiments, the divalent cation is selected from Ca²⁺, Ba²⁺, Mg²⁺ or Mn²⁺, in particular Ca²⁺. In certain independent embodiments, the salt is CaCl₂, the zwitterionic buffer is HEPES, and the anionic small molecule drug is MTX or DOX.

Examples of zwitterionic buffers, also known as Good buffers, appropriate for keeping a physiological pH and for use in accordance with the methods of the present invention are well known to the person skilled in the art according to their accepted acronym or common name: MES, ADA, PIPES, ACES, MOPSO, Cholamine Chloride, MOPS, BES, TES, HEPES, DIPSO, Acetamidoglycine, TAPSO, POPSO, HEPPSO, HEPPS, Tricine, Glycinamide, Bicine, TAPS, AMPSO, CABS, CHES, CAPS and CAPSO.

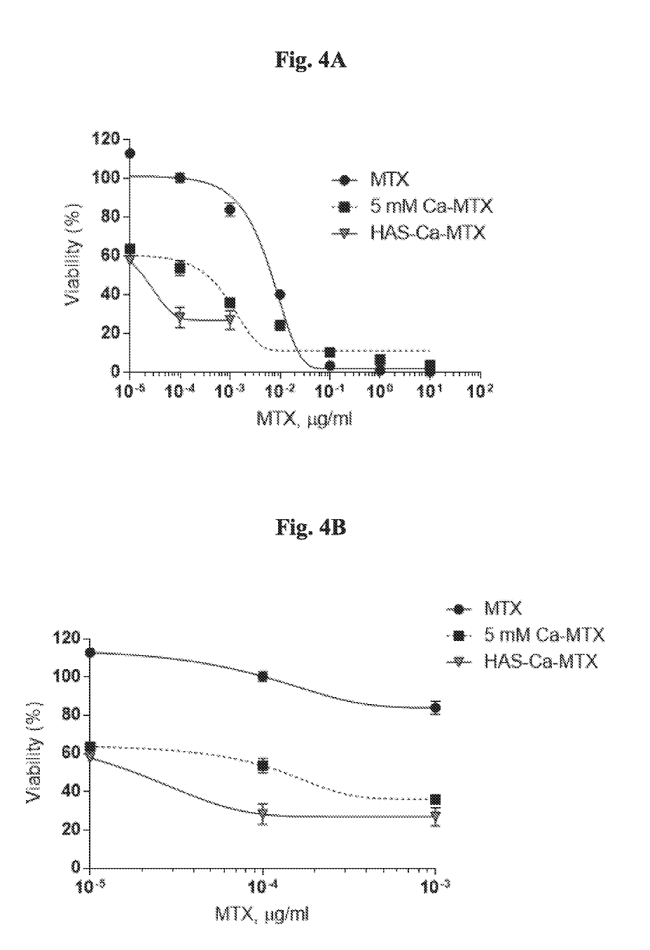

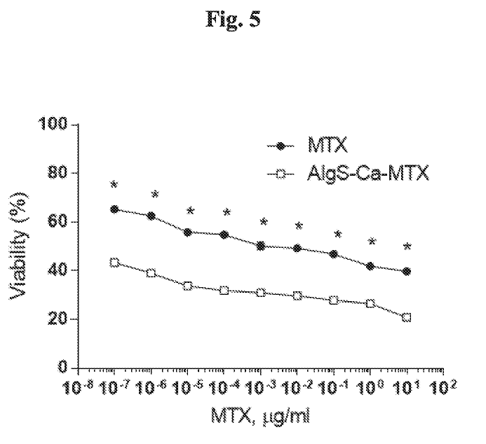

The present inventors have further found that in addition to nanoparticles described above, also nanoparticles lacking an anionic polymer, thereby comprising complexes of an anionic small molecule drug and a calcium ion, are stable, have a negative surface charge, and are capable of entering into cells and affecting their viability (see Example 2, FIG. 4).

Similar to the nanoparticles containing an anionic polymer described above, these nanoparticles are also generally of a similar size and surface charge as the nanoparticles containing an anionic polymer.

The inventors found that, in contrast to complexes formed with calcium phosphate (a weak electrolyte) that results in uncontrollable growth of calcium phosphate crystals in physiological solutions that could result in significant cytotoxicity, the complex formed with CaCl₂ (a salt that is a strong electrolyte with close to complete dissociation of the ions in water), forms nanoparticles (NPs) and a colloid suspension when mixed with an aqueous solution.

Thus, in yet a further aspect, the present invention provides an anionic nanoparticle comprising a divalent cation, which is derived or dissociated from a strong electrolyte, and an anionic small molecule drug and lacking an anionic polymer, wherein said nanoparticle is in the form of nanoparticles and capable of forming a colloidal suspension as measured by dynamic light scattering (DLS). The term “colloidal suspension refers to a suspension in which the nanoparticles do not precipitate or sink to the bottom of the vehicle holding the solution. The source of the calcium in the complex is not calcium phosphate and therefore the complex is essentially lacking phosphate ions. The present invention further provides a method for producing the complex (comprising a divalent cation and an anionic small molecule drug and lacking an anionic polymer), comprising mixing said anionic small molecule drug with a salt of a divalent cation that is a strong electrolyte in zwitterionic buffer at physiological pH. Thus, it is evident that the method for producing the complex comprising a divalent cation and an anionic small molecule drug and lacking an anionic polymer, could be considered a first step in the method for producing the complex described above.

In certain embodiments, the divalent cation is selected from Ca²⁺, Ba²⁺, Mg²⁺ or Mn²⁺, in particular Ca²⁺. In certain independent embodiments, the salt is CaCl₂, the zwitterionic buffer is HEPES, and the anionic small molecule drug is MTX or DOX. Examples of additional drugs that may be used in accordance with the present invention are listed above in the context of the nanoparticles which include an anionic polymer.

In certain embodiments, the final Ca²⁺ concentration around the cells is above 3 mM, and in particular is above 2.5 mM. In certain embodiments, the calcium concentration is about 5 mM.

The terms “strong electrolyte” and “physiological pH” are as defined herein above.

The anionic nanoparticles of the invention, which lack an anionic polymer, may be used for delivery of small anionic drug molecules into cells, and/or for treating a disease or disorder or condition in a subject.

In some embodiments, the disease or disorder or condition is selected from colon cancer, ovarian carcinoma, and breast cancer, or metabolic, neurodegenerative, cardiovascular, infectious or inflammatory diseases or disorders. In some embodiments the disease disorder or condition is cancer

The invention will now be illustrated by the following non-limitative examples. [see if we want to add a size range and a range of zeta potential (also for the first complex)]

### EXAMPLES

### Experimental

### Materials:

Hyaluronan (sodium salt, 150 kDa) was from Lifecore Biomedical, Chaska, Minn. Alginate (30-50 kDa) was purchased from NovaMatrix. Hyaluronan-sulfate (HAS) and alginate-sulfate (AlgS) were prepared as previously described (Freeman et al., 2008, Biomaterials 29, 3260-8). Gold labeling of HAS and AlgS with Monoamino Nanogold® labeling reagent (NH₂—Au, mean diameter 1.4 nm) (Nanoprobes, Yaphank, N.Y.) was performed using carbodiimide chemistry as previously described (Polyak, et al., 2004, Biomacromolecules 5, 389-396). Cell culture reagents: Dulbecco's modified Eagle's medium (DMEM), RPMI1640, L-glutamine, penicillin/streptomycin, heat inactivated Foetal Bovine Serum (FBS) were from Biological Industries (Kibbutz Beit-Haemek, Israel). Doxorubicin-HCl (DOX) was from Ebewe Pharma (Unterach, Austria). Methotrexate (MTX) and other reagents, unless specified otherwise, were from Sigma. All reagents were of analytical grade.

### Cell Lines

Mouse colon carcinoma CT26 cell line purchased from the American Type Culture Collection (ATCC, Rockville, Md.) were cultured in DMEM supplemented with 10% FBS, 2 mM L-glutamine, and 1% penicillin/streptomycin.

MDA-MB-231 human breast cancer cell line was purchased from American Type Culture Collection (ATCC, Rockville, Md.) and cultured in RPMI1640 supplemented with 10% FBS, 2 mM L-glutamine, and 1% penicillin/streptomycin.

NCI-ADR/Res (NAR) human ovary carcinoma cell line (DOX-resistant) was purchased from American Type Culture Collection (ATCC, Rockville, Md.) and cultured in RPMI1640 supplemented with 10% FBS, 2 mM L-glutamine, and 1% penicillin/streptomycin.

### Preparation and Characterization of MTX and DOX Nanoparticles (NPs)

Preparation of MTX Nanoparticles

Equal volumes of MTX and CaCl₂ (both at stock concentrations as detailed in Table 1 and Table 2) were mixed together by vortexing for 30 sec and were incubated for 20 min at room temperature (RT) to allow complex formation. Then, equal volumes of the HAS or AlgS (50 μg/mL in 10 mM HEPES) or 10 mM HEPES were added and mixed by vortexing for 30 sec, followed by 30 min incubation at RT prior to use. The final concentrations of each component in the formulations used in the study, after diluting 1:50 in 10 mM HEPES, are detailed in Table 1 and Table 2.

TABLE 1
Summary of stock and final component concentrations of
Ca²⁺-MTX and HAS-Ca²⁺-MTX formulations used in the study.

| [MTX], μg/ml | [Ca²⁺], M | [HAS], μg/ml |
|---|---|---|
| Stock/final | Stock/final | Stock/final |
| 2 × 10³/10 | 1/5 × 10⁻³ | |
| 200/1 | 1/5 × 10⁻³ | |
| 20/0.1 | 1/5 × 10⁻³ | |
| 2/1 × 10⁻² | 1/5 × 10⁻³ | |
| 0.2/1 × 10⁻³ | 1/5 × 10⁻³ | 50/0.5 or 0 |
| | 0.2/1 × 10⁻³ | |
| 2 × 10⁻²/1 × 10⁻⁴ | 1/5 × 10⁻³ | 50/0.5 or 0 |
| | 0.2/1 × 10⁻³ | |
| 2 × 10⁻³/1 × 10⁻⁵ | 1/5 × 10⁻³ | 50/0.5 or 0 |
| | 0.2/1 × 10⁻³ | |

TABLE 2
Summary of stock and final component concentrations
of AlgS-Ca²⁺-MTX formulations used in the study.

| [MTX] (μg/ml), | [CaCl₂] (M), | [AlgS] (μg/ml), |
|---|---|---|
| stock/final | stock/final | stock/final |
| 2 × 10³/10 | 1/5 × 10⁻³ | 50/0.5 |
| 200/1 | 1/5 × 10⁻³ | 50/0.5 |
| 20/0.1 | 1/5 × 10⁻³ | 50/0.5 |
| 2/1 × 10⁻² | 1/5 × 10⁻³ | 50/0.5 |
| 0.2/1 × 10⁻³ | 1/5 × 10⁻³ | 50/0.5 |
| 2 × 10⁻²/1 × 10⁻⁴ | 1/5 × 10⁻³ | 50/0.5 |
| 2 × 10⁻³/1 × 10⁻⁵ | 1/5 × 10⁻³ | 50/0.5 |
| 2 × 10⁻⁴/1 × 10⁻⁶ | 1/5 × 10⁻³ | 50/0.5 |
| 2 × 10⁻⁵/1 × 10⁻⁷ | 1/5 × 10⁻³ | 50/0.5 |

Preparation of DOX Nanoparticles

Equal volumes of DOX and CaCl₂ (both at stock concentrations as detailed in Table 3) were mixed together by vortexing for 30 sec and were incubated for 20 min at room temperature (RT) to allow complex formation. Then, equal volumes of AlgS (50 μg/mL in 10 mM HEPES) were added and mixed by vortexing for 30 sec, followed by 30 min incubation at RT prior to use. The final concentrations of each component in the NPs used in the study, after diluting 1:50 in 10 mM HEPES, are detailed in Table 3.

TABLE 3
Summary of stock and final component concentrations
of AlgS-Ca²⁺-DOX formulations used in the study.

| DOX (μg/ml), | CaCl₂ (M), | AlgS (μg/ml), |
|---|---|---|
| stock/final | stock/final | stock/final |
| 2 × 10³/10 | 1/5 × 10⁻³ | 50/0.5 |
| 2 × 10³/5.8 | 1/5 × 10⁻³ | 50/0.5 |
| 200/0.58 | 1/5 × 10⁻³ | 50/0.5 |
| 20/5.8 × 10⁻² | 1/5 × 10⁻³ | 50/0.5 |
| 2/5.8 × 10⁻³ | 1/5 × 10⁻³ | 50/0.5 |
| 0.2/5.8 × 10⁻⁴ | 1/5 × 10⁻³ | 50/0.5 |
| 2 × 10⁻²/5.8 × 10⁻⁵ | 1/5 × 10⁻³ | 50/0.5 |
| 2 × 10⁻³/5.8 × 10⁻⁶ | 1/5 × 10⁻³ | 50/0.5 |
| 2 × 10⁻⁴/5.8 × 10⁻⁷ | 1/5 × 10⁻³ | 50/0.5 |

Particle Size and Zeta (ζ) Potential Measurements

Particle size distribution and mean diameter of MTX and DOX anionic nanoparticle complexes were measured on a CGS-3 (ALV, Langen, Germany) instrument. Samples were diluted 1:50 in 10 mM HEPES solution and were analyzed by scattered laser light (He—Ne laser, 20 mW, 632.8 nm) and detected under an angle of 90°, during 10 s for 20 times, at 25° C. Correlograms were calculated by ALV/LSE 5003 correlator and fitted with version of the program CONTIN. The surface charge (ζ potential, mV) of the complexes was measured on a Zetasizer Nano ZS (Malvern Instruments Ltd., UK) using electrophoretic cells (DTS 1060, produced by Malvern Instruments Ltd., UK). Zeta potentials were recorded three times, 10 to 100 measurements in each run (depending on standard deviation).

Transmission Electron Microscopy

MTX complexes (with or w/o gold-labeled HAS, at final concentrations of: MTX—5 ng/ml, Ca²⁺—250 mM, HAS—25 μg/ml, all in HEPES 10 mM) and DOX NPs (with gold-labeled AlgS, at final concentrations of: DOX—0.58 μg/ml, Ca²⁺— 250 mM, AlgS—25 μg/ml, all in HEPES 10 mM) were prepared as described above.

5 μL of each sample were placed on carbon-coated films on copper EM grids hydrophilized by glow discharge. The excess liquid was blotted and the grids were allowed to dry at RT overnight. The samples were imaged at RT using a FEI Tecnai 12 G² TWIN TEM (Gatan model 794 CCD, bottom mounted) at acceleration voltage of 120 kV. Specimens were studied in a low-dose imaging mode to minimize beam exposure and electron beam radiation damage. Images were recorded digitally using the Digital Micrograph 3.6 software (Gatan, Munich, Germany).

### Cellular Uptake of Free DOX and AlgS-Ca²⁺-DOX NPs

Imaging experiments using an imaging flow cytometer (Amnis ImageStreamX, ISX) were performed to quantitatively evaluate the cellular uptake of DOX. Cells were seeded in 6-well culture plates at a cell density of 300,000 cells per well. Twenty-four hours post-seeding, the cells were incubated for 4 h with Free DOX or AlgS-Ca²⁺-DOX anionic NPs (5.8 μg/ml DOX, 5 mM Ca²⁺, 0.5 μg/ml AlgS), and then medium was replaced to fresh medium for 24 h. For ISX analysis, the cells were harvested (at 4 or 24 h after DOX NP addition) by trypsinization followed by centrifugation at 300 g for 5 min. The cell pellet was resuspended in FACS buffer (PBS containing 2% FBS, v/v) and cells were analyzed using ImageStreamX Mark II (Amnis, Seattle, Wash.). Cell acquisition and analysis were performed using IDEAS Application, version 6.0.

### In Vitro Cytotoxicity

MTX Nanoparticles

CT26 mouse colon carcinoma cells or MDA-MB-231 human breast cancer cell line were seeded in 48-well plates at a density of 10,000 cells per well. After 24 h, medium was replaced with medium containing MTX nano-complexes or free MTX at various concentrations. After 48 h incubation, cell viability was assessed using PrestoBlue cell viability assay (Life Technologies, Carlsbad, Calif.). The assay is based on the live cell's ability to reduce resazurin (non-fluorescent) to resorufin (fluorescent). PrestoBlue working solution was prepared by dilution of PrestoBlue reagent 1:10 in cell culture medium. PrestoBlue working solution was added to the cells for 1 h at 37° C. and 5% CO₂. Fluorescent intensity was measured using the Synergy Mx microplate reader (Biotek, Winooski, Vt.) at an excitation wavelength of 560 nm and emission wavelength of 590 nm. The percentage of cell viability was obtained after normalizing the data to untreated cells.

DOX Nanoparticles

MDA-MB-231 or DOX-resistant human ovary carcinoma cell line NAR cells were seeded in 48-well plates at a density of 10,000 cells per well. After 24 h, medium was replaced with medium containing DOX NPs or free DOX at various concentrations. After incubation for 4 h, the medium was replaced to drug-free culture medium. After 48 h incubation, cell viability was assessed using PrestoBlue cell viability assay, as described above.

### Statistical Analysis

Statistical analysis was performed with GraphPad Prism version 6.05 for Windows (GraphPad Software, San Diego, Calif.). All variables are expressed as mean±SEM. To test the hypothesis that changes in DLS and potential measurements varied over time among the experimental groups, a general linear 2-way repeated-measures (RM) ANOVA model was used. The model included the effects of treatment, time, and treatment-by-time interaction, with Sidak's multiple comparisons test. Viability data was fit using 4-parametric logistic regression curve, where applicable. IC₅₀ values obtained from the intersection of regression line with 50% response. Differences between experimental groups as a function of drug concentration were evaluated by 2-way ANOVA, with with Sidak's multiple comparisons test. P<0.05 was considered statistically significant.

### Example 1: MTX Complexes Physical Characterization

MTX Complex Optimization and Stability

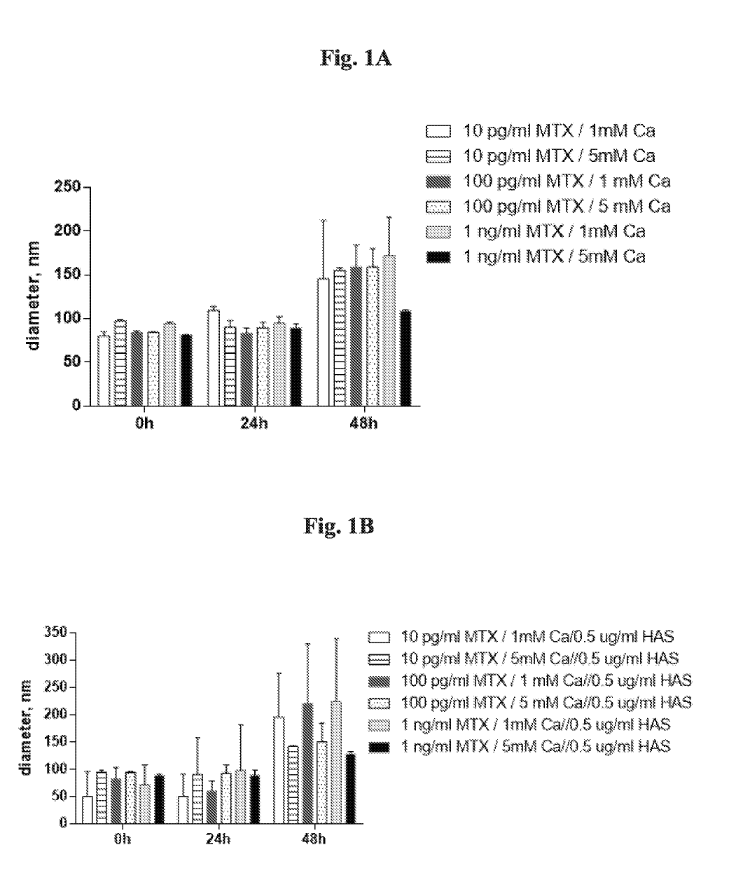

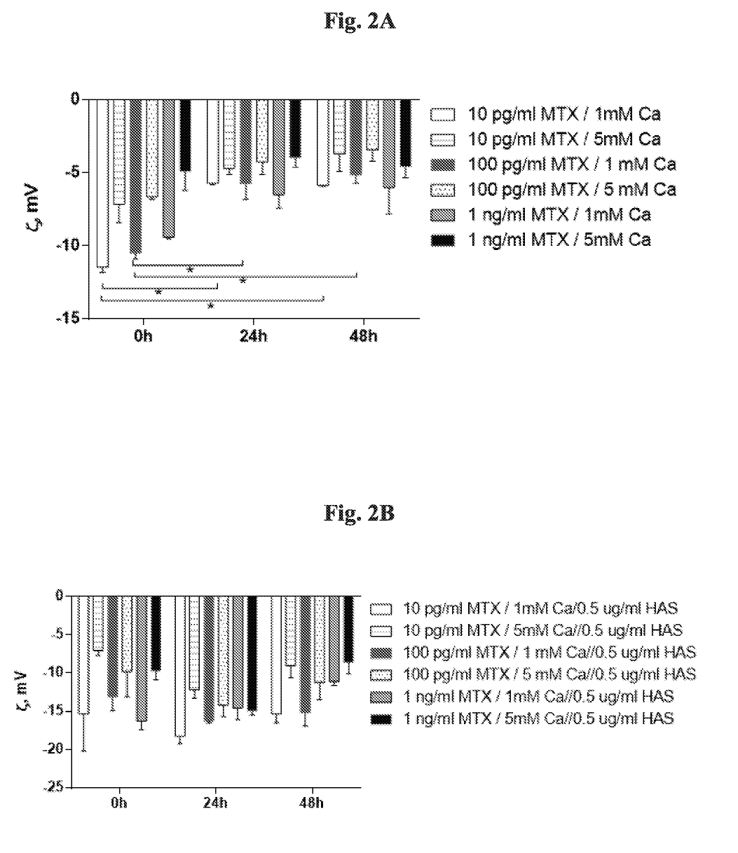

DLS measurements showed the formation of nano-sized complexes between calcium and MTX, of ˜100 nm in diameter (FIG. 1A). There was a trend toward increase in complex diameter with time at lower calcium and MTX concentrations, while 5 mM Ca²+/1 ng/ml MTX showed preserved size even after 48 h. Similar DLS results were also observed with HAS-Ca²⁺-MTX complexes (FIG. 1B). ζ potential measurements showed reduction in surface charge with the increase in calcium ion/MTX concentration, as a function of time (FIG. 2A). Presence of HAS in the complexes improved their overall stability as ζ potential measurements did not reveal and significant changes in the surface charge over time (FIG. 2B).

MTX Complexes with AlgS

DLS measurements showed the formation of nano-sized complexes between calcium MTX, and AlgS of ˜100 nm in diameter, similar to the results that were observed with HAS-Ca²⁺-MTX complexes. ζ potential measurements showed reduction in surface charge with increased MTX concentrations. Surface charge measurements of AlgS-Ca²⁺-MTX complexes showed similar values to HAS-Ca²⁺-MTX complexes prepared with same concentration of CaCl₂ (5 mM Ca²⁺ final concentration, data not shown).

MTX Complex Analysis by Electron Microscopy

The formation of nano-sized complexes was confirmed by dry transmission electron microscopy (TEM) (FIG. 3). Dry-TEM analysis showed compact and nearly spherical Ca²⁺-MTX complexes. The introduction of gold-labeled HAS resulted in the formation of similar-sized complexes, but with morphologically distinct organization, showing fiber-like internal structures, attributed to the presence of the polymer. The observed sizes of the resulting complexes were comparable with DLS measurements.

### Example 2: In Vitro Anti-Tumor Efficacy of MTX NPs in CT26 Cells

After 48 h incubation with various MTX complexes, the viability profile of CT26 mouse colon carcinoma was determined (FIG. 4). The obtained half maximal inhibitory concentration (IC₅₀) values for free MTX, Ca²⁺-MTX (5 mM Ca²⁺), and HAS-Ca²⁺-MTX (5 mM Ca²⁺, 0.5 μg/ml HAS) complexes are shown in Table 4. Both MTX formulations exhibited greater cytotoxicity vs. free drug, with HAS-Ca²⁺-MTX complexes being the most effective (˜10-fold and ˜400-fold, vs. Ca²⁺-MTX and free MTX, respectively).

TABLE 4
Comparative IC₅₀ values of free MTX, and MTX
nano-complexes in CT26 cell line (μg/ml).

| | | 0.5 μg/ml HAS/5 mM |
|---|---|---|
| MTX | 5 mM Ca²⁺/MTX | Ca²⁺/MTX |
| 6.96 × 10⁻³ | 1.36 × 10⁻⁴ | 1.79 × 10⁻⁵ |

### Example 3: In Vitro Anti-Tumor Efficacy of MTX NPs in MDA-MB-231 Cells

After 48 h incubation with various MTX complexes, the viability profile of MDA-MB-231 human breast cancer cell line was determined (FIG. 5). At all concentrations tested, treatment with AlgS-Ca²⁺-MTX NPs (0.5 μg/ml AlgS, 5 mM Ca²⁺) resulted in significantly reduced cell viability (e.g., increased cytotoxicity) (p, interaction, 2-way ANOVA<0.0001).

### Example 4: DOX NP Preparation and Physical Characterization

DOXNPs with AlgS

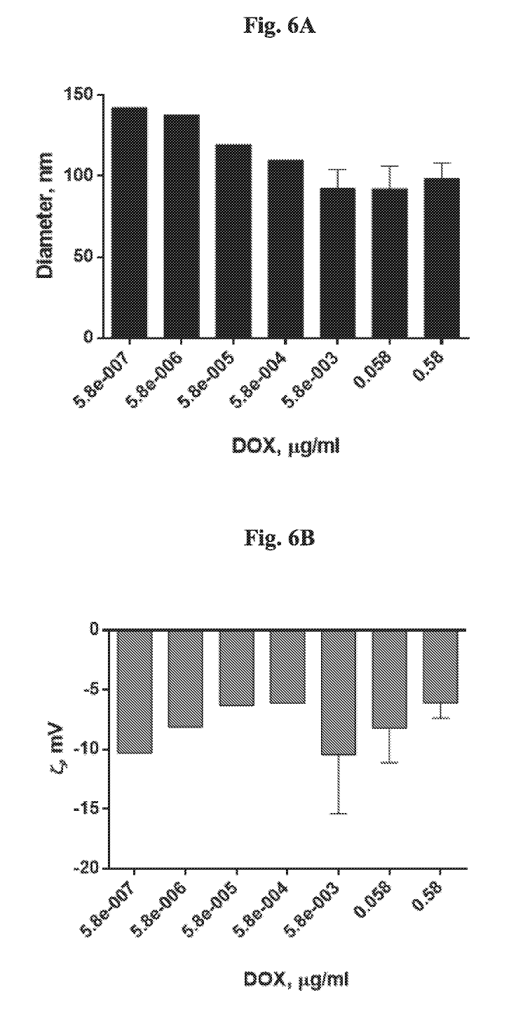

AlgS-Ca²⁺-DOX complexes were prepared as listed in Table 3. DLS measurements showed formation of ˜100 nm diameter, nano-sized complexes (FIG. 6A). A slight decrease in size was observed with increasing concentrations of DOX. Surface charge analysis of AlgS-Ca²⁺-DOX NPs by ζ potential measurements showed slightly negative surface charge with a range of −10 to −6 mV (FIG. 6B).

DOXNP Analysis by Electron Microscopy

Dry-TEM analysis using gold-labeled AlgS showed compact and nearly spherical AlgS-Ca²⁺-DOX NPs (FIG. 7). The observed sizes of the resulting NPs were comparable with DLS measurements.

### Example 5: In Vitro Anti-Tumor Efficacy of DOX NPs

MDA-MB-231 Cells

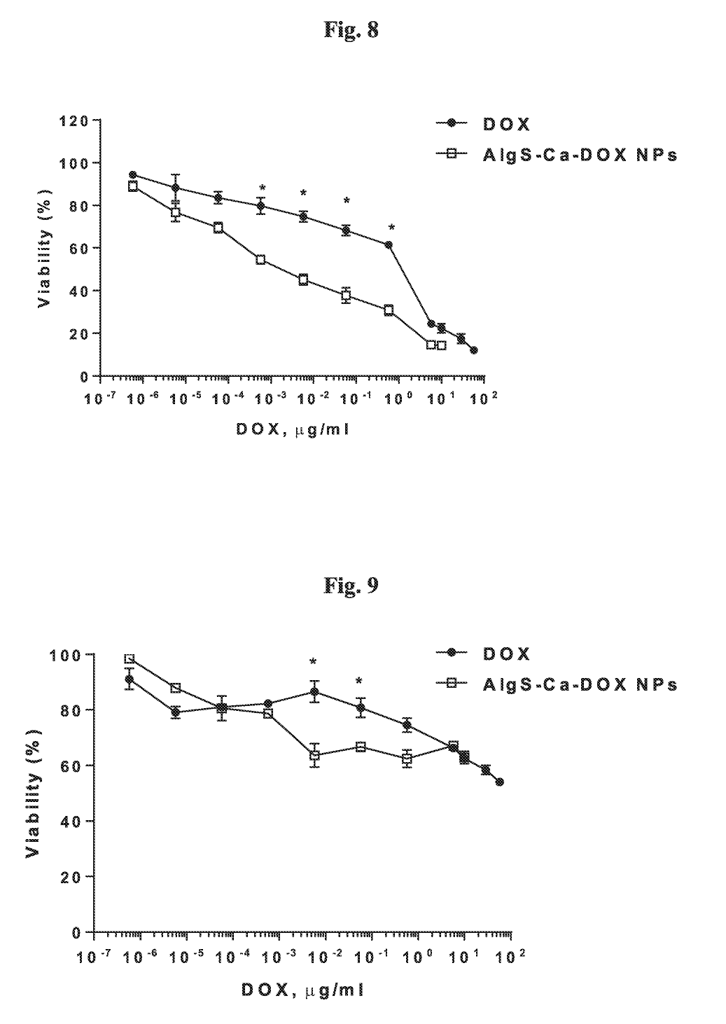

After 48 h incubation with various DOX complexes, the viability profile of MDA-MB-231 human breast cancer cell line was determined (FIG. 8). Treatment with AlgS-Ca²⁺-DOX NPs resulted in significantly reduced cell viability (e.g., increased cytotoxicity) (p, interaction, 2-way ANOVA<0.0001). The respective half maximal inhibitory concentration (IC₅₀) values were 1.27 μg/ml for free MTX, and 1.68×10⁻³ μg/ml, for AlgS-Ca²⁺-DOX NPs.

NAR Cells

After 48 h incubation with various DOX complexes, the viability profile of multidrug resistant human ovarian carcinoma cell line, (NCI-ADR/Res (NAR)), overexpressing the P-glycoprotein (P-gp) extrusion pump was determined (FIG. 9). The treatment with AlgS-Ca²⁺-DOX NPs resulted in significantly reduced cell viability (e.g., increased cytotoxicity) at 5.8×10⁻³-5.8×10⁻² μg/ml concentration range (p, interaction, 2-way ANOVA=0.0013). The low response of the cells to the treatment could be explained by extrusion of the drug by this drug-resistant cell line, as the anionic NP delivery system is not designed to actively interfere with the extrusion mechanism.

### Example 6: Cellular Uptake of Free DOX and AlgS-Ca²⁺-DOX NPs

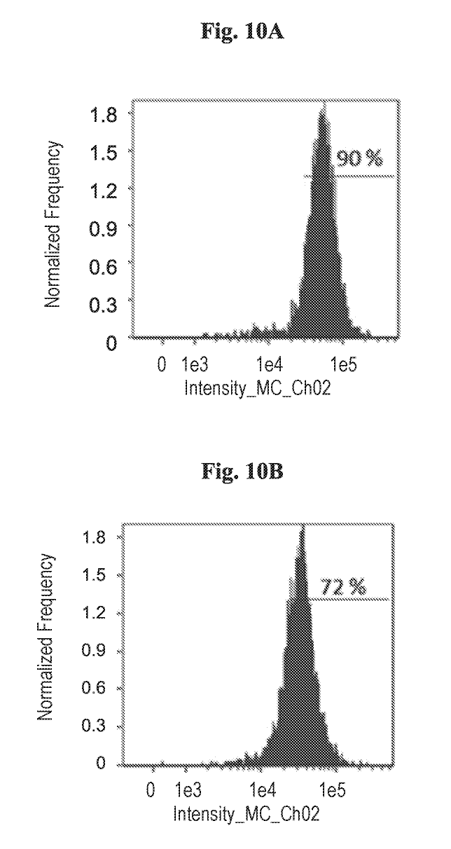

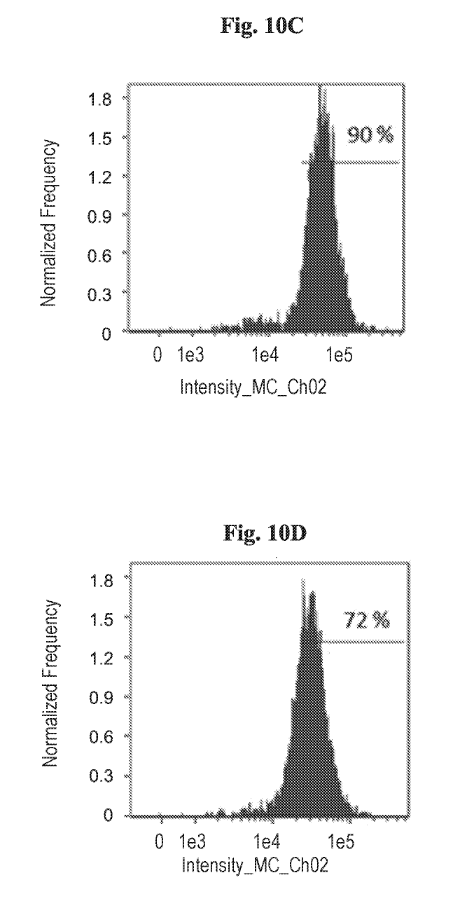

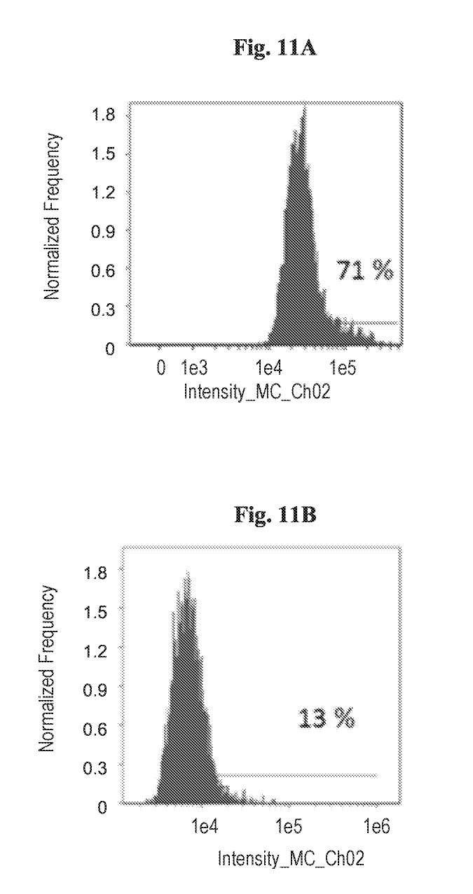

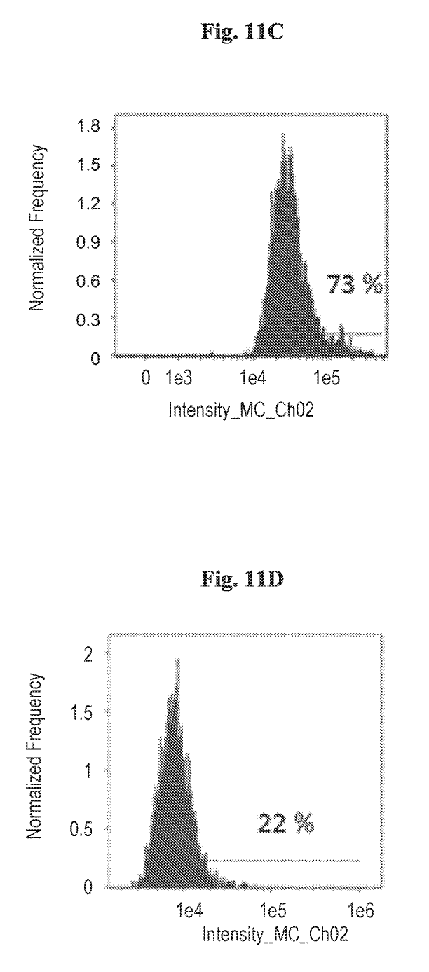

Using the Amnis ImageStreamX we took advantage of the fluorescent properties of DOX in order to follow the cellular uptake and intracellular accumulation (Free DOX vs. AlgS-Ca²⁺-DOX NPs at 5.8 μg/ml DOX) in MDA-MB-231 and NAR cells (FIGS. 10 and 11). In both cell types the fluorescence intensity at 4 h and 24 h post treatment was similar with Free DOX compared to the NPs (Table 5). In MDA-MB-231 cells, the results quantitatively showed similar DOX intracellular accumulation at 4 h post-treatment (90%), with modest decrease to 72% at 24 h post exposure in Free DOX and NP samples, as calculated by the percentage of cells that were positive for DOX.

A multidrug resistant human ovarian carcinoma cell line, (NCI-ADR/Res (NAR)), overexpressing the P-glycoprotein (P-gp) extrusion pump, was selected to test the ability of AlgS-Ca²⁺-DOX NPs to overcome drug resistance. While at 4 h post-treatment both free DOX and NP samples showed similar uptake values (˜70%), at 24 h markedly lower values of fluorescence intensity and accumulation of DOX in cells were observed. Importantly, the accumulation of DOX was higher with AlgS-Ca²⁺-DOX NPs vs. Free DOX (22% vs. 13%, respectively). These results suggest that the DOX encapsulated in AlgS-Ca²⁺-DOX NPs might not be detoxified and extruded as quickly as free DOX by P-gp, and the drug could be retained for a longer time in the resistant cells after internalization. These results support the partial response of the cells to the drug when delivered by NPs at lower drug concentrations that was observed in cytotoxicity study.

TABLE 5
Cellular uptake analysis data in MDA-MB-231 and NAR cells,
as assessed by ImagestreamX imaging flow cytometer.

| | Treatment | Mean | |
|---|---|---|---|
| | (time post | Fluorescent | |
| Cell type | transfection) | intensity | % positive cells |
| MDA-MB 231 | Free DOX (4 h) | 59061 | 90% |
| | AlgS-Ca²⁺-DOX (4 h) | 54894 | 90% |
| | Free DOX (24 h) | 41944 | 72% |
| | AlgS-Ca²⁺-DOX (24 h) | 39097 | 72% |
| NAR | Free DOX (4 h) | 40932 | 71% |
| | AlgS-Ca²⁺-DOX (4 h) | 43539 | 73% |
| | Free DOX (24 h) | 15108 | 13% |
| | AlgS-Ca²⁺-DOX (24 h) | 15390 | 22% |

### Example 7: In Vivo Antitumor Activity of AlgS-Ca²⁺-DOX NPs

Female athymic nude mice (six weeks old, body weight=20-23 g) are inoculated to the right cranial trunk region with MDA-MB-231 cells/Matrigel™ 1:1 (v/v) suspension (3×10⁶ cells/0.2 mL/animal). Treatment is commenced when tumor size is approximately 100-150 mm³. Mice are treated intravenously (i.v.) with three weekly injections of 4 mg/kg (weekly cumulative dose 12 mg/kg) of DOX or DOX NPs in saline. NPs were prepared as described above using the following stock solutions: 2 mg/ml DOX, 1M CaCl₂, 50 μg/ml AlgS. Prior to injection: DOX (0.5 mg/ml), or DOX NPs were diluted 1:1.5 in saline. Tumor progression is monitored twice a week by caliper measurement and calculated using the formula: Volume=(width)²×length/2. Mice are observed daily, and body weight is detected as possible signs of toxicity. For histological analysis, mice are euthanized at the end of the treatments. Following excision, tumors are weighted, fixed in 4% formaldehyde, embedded in paraffin, and sectioned into 5-μm slices. Tissue samples are analyzed by H&E staining for histopathological changes, Ki67 staining for cell proliferation and TUNEL assay for apoptotic cell death.

It is expected that the tumor size will be reduced in the DOX-treated group, and more significantly reduced in the group treated with DOX complexes, compared to controls. Further, the DOX-related cardiotoxic effect is expected to be reduced in the DOX NPs treated mice compared with the DOX-treated mice.

### REFERENCES

Barenholz, Y., Doxil®—The first FDA-approved nano-drug: lessons learned. J. Control. Release 160, 117-134 (2012).
Bertrand, N., Wu, J., Xu, X., Kamaly, N. & Farokhzad, O. C. Cancer nanotechnology: the impact of passive and active targeting in the era of modern cancer biology. Adv. Drug Deliv. Rev. 66, 2-25 (2014).
Blanco E, Shen H & Ferrari M Principles of nanoparticle design for overcoming biological barriers to drug delivery, Nature Biotechnology 33(9), 941-951 (2015).
Maeda, H., Nakamura, H. & Fang, J. The EPR effect for macromolecular drug delivery to solid tumors: Improvement of tumor uptake, lowering of systemic toxicity, and distinct tumor imaging in vivo. Adv. Drug Deliv. Rev. 65, 71-79 (2013).

1. An anionic nanoparticle formed by electrostatic interactions at aqueous conditions from an anionic polymer, an anionic small molecule drug, and a cation, wherein said anionic polymer is an anionic natural polysaccharide or a derivative thereof, or an anionic synthetic polymer.

2. The anionic nanoparticle of claim 1, wherein said anionic polymer includes an anionic natural polysaccharide or a derivative thereof and said anionic natural polysaccharide is alginate (Alg) or hyaluronic acid (HA), and said derivative is alginate sulfate (AlgS) or hyaluronan sulfate (HAS).

3. The anionic nanoparticle of claim 1, wherein said anionic polymer comprises a targeting moiety.

4. The anionic nanoparticle of claim 1, wherein said cation is a divalent cation.

5. The anionic nanoparticle of claim 4, wherein said divalent cation is Ca²⁺.

6. The anionic nanoparticle of claim 1, wherein said anionic small molecule drug is selected from the group consisting of methotrexate (MTX), doxorubicin (DOX), carboxylate derivatives of taxol and camptothecin, flavopiridol, imatinib, phenobarbital and barbituric acid, valproate, furosemide, salicylate, acetylsalicylate, probenecid, bumetanide, piroxicam, azidodeoxythymidine, benzylpenicillin, AMD3100 (plerixafor), and an alkyl sulfonate.

7. The anionic nanoparticle of claim 6, wherein said anionic small molecule drug is MTX or DOX.

8. The anionic nanoparticle of claim 1, wherein the anionic nanoparticle is selected from the group consisting of an Alg-Ca²⁺-MTX nanoparticle, an AlgS-Ca²⁺-MTX nanoparticle, a HA-Ca²⁺-MTX nanoparticle, a HAS-Ca²⁺-MTX nanoparticle, an Alg-Ca²⁺-DOX nanoparticle, an AlgS-Ca²⁺-DOX nanoparticle, a HA-Ca²⁺-DOX nanoparticle, and a HAS-Ca²⁺-DOX nanoparticle.

9. The anionic nanoparticle of claim 8, wherein the anionic nanoparticle is a HAS-Ca²⁺-MTX nanoparticle, an AlgS-Ca²⁺-MTX, or an AlgS-Ca²⁺-DOX nanoparticle.

10.-11. (canceled)

12. A method of treating a disease, disorder or condition in a subject in need thereof comprising administering to said subject an anionic nanoparticle formed by electrostatic interactions at aqueous conditions from an anionic polymer, an anionic small molecule drug, and a cation, wherein said anionic polymer is an anionic natural polysaccharide or a derivative thereof, or an anionic synthetic polymer, wherein said disease, disorder or condition is selected from the group consisting of metabolic, neurodegenerative, cardiovascular, infectious, and inflammatory diseases or disorders, and cancer.

13. The method of claim 12, wherein said disease, disorder or condition is cancer.

14. A method for preparing an anionic nanoparticle formed by electrostatic interactions at aqueous conditions from an anionic polymer, an anionic small molecule drug, and a cation, wherein said anionic polymer is an anionic natural polysaccharide or a derivative thereof, or an anionic synthetic polymer, comprising mixing said anionic small molecule drug with a salt of a divalent cation that is a strong electrolyte in a zwitterionic buffer at a physiological pH, and adding said anionic polymer.

15.-16. (canceled)

17. The method of claim 14, wherein said divalent cation is selected from the group consisting of Ca²⁺, Ba²⁺, Mg²⁺ and Mn²⁺.

18. The method of claim 17, wherein said divalent cation is Ca²⁺.

19. The method of claim 18, wherein said salt is CaCl₂) and said zwitterionic buffer is HEPES.

20. (canceled)

21. The anionic nanoparticle of claim 4, wherein said divalent cation is selected from the group consisting of Ca²⁺, Ba²⁺, Mg²⁺ and Mn²⁺.
